(19) United States
(12) Patent Application Publication
(10) Pub. No.: US 2026/0013969 A1
(43) Pub. Date: Jan. 15, 2026

(54) Wireless Augmented Reality Eyewear System for Real-Time Medical Visualization

(71) Applicant: Jorge Gonzalez, Fort Lauderdale, FL (US)

(72) Inventor: Jorge Gonzalez, Fort Lauderdale, FL (US)

(21) Appl. No.: 19/254,356

(22) Filed: Jun. 30, 2025

Related U.S. Application Data
(60) Provisional application No. 63/669,707, filed on Jul. 11, 2024.

(51) Int. Cl. A61B 90/00; A61B 90/50; G02B 27/01; G09G 3/00

(52) U.S. Cl. CPC A61B 90/37; G02B 27/0172; G09G 3/001; A61B 2090/365; A61B 2090/372; A61B 2090/502; G02B 2027/0178; G09G 2320/0626; G09G 2360/144; G09G 2380/08

(57) ABSTRACT

A wireless augmented reality (AR) system for use by medical practitioners during diagnostic, surgical, or interventional procedures is disclosed. The system includes AR eyewear with integrated heads-up displays (HUDs) embedded in semi-transparent optical lenses, enabling video content to be projected without obstructing the user's natural field of view. Each lens includes a high-resolution micro display and an ambient light sensor for automatic brightness adjustment. The eyewear is coupled via a wired connection to a portable control unit containing a microprocessor for real-time decoding and formatting video signals. A wireless transmitter connects to a medical video source and transmits the live feed to the control unit via a high-bandwidth wireless protocol. The system enables projection of anatomical overlays in real-time, supporting visualization of muscle tissue, nerves, tendons, connective structures, and any other human or animal body part.

### CROSS-REFERENCE TO RELATED APPLICATION

The present application claims priority to, and the benefit of, U.S. Provisional Application No. 63/669,707 which was filed on Jul. 11, 2024 and is incorporated herein by reference in its entirety.

### FIELD OF THE INVENTION

The present invention generally relates to the field of medical technology and wearable visualization systems. More specifically, the present invention relates to a wireless augmented reality (AR) system configured for real-time, ergonomic, and hands-free display of medical imaging data during diagnostic and surgical procedures. The system comprises a pair of AR eyewear with integrated heads-up displays (HUDs) that project video feeds from medical instruments directly into the user's field of view, thereby eliminating the need to look away from the patient. The eyewear features high-resolution micro displays, ambient light sensors for automatic brightness adjustment, and a semi-transparent lens design that maintains situational awareness. A portable control unit receives video wirelessly from a medical imaging source, decodes the video, and transmits the display feed to the eyewear via, for example, a USB-C connection. Accordingly, the present disclosure makes specific reference thereto. Nonetheless, it is to be appreciated that aspects of the present invention are also equally applicable to other like applications, devices, and methods of manufacture.

### BACKGROUND

By way of background, in various medical and veterinary procedures, including diagnostic and interventional techniques such as colonoscopies, endoscopies, ultrasounds, and laparoscopies, require real-time visualization of internal anatomy. Such procedures are commonly performed using video-equipped medical instruments that transmit visual data to external monitors. The monitors are usually mounted on carts or workstations positioned at a distance from the practitioner. As a result, the medical practitioner is often required to divert their gaze from the operative or diagnostic field to view the video feed, which may be located above eye level or to the side of the working area.

Accordingly, the medical practitioners have to do repetitive head movement, eye refocusing, and unnatural postures, which can lead to increased neck strain and visual fatigue. Furthermore, the physical separation between the practitioner and the display monitor can compromise procedural efficiency, increase the risk of errors, and contribute to musculoskeletal disorders due to prolonged or awkward body positioning.

During procedures that involve real-time image guidance, practitioners may also need to adjust or reposition the patient or the imaging equipment itself to optimize the view, which adds to the physical demands of the task. The video feeds are displayed on conventional monitors and are often small or of limited resolution, requiring practitioners to squint or lean in to discern anatomical details, further increasing eye strain and reducing comfort. Accordingly, individuals desire an ergonomic visualization system that enables practitioners to receive real-time video data directly within their line of sight, thereby eliminating the need for constant head turning or repositioning and improving both procedural accuracy and practitioner well-being.

Therefore, there exists a long-felt need in the art for a wireless augmented reality (AR) system that overcomes the limitations of conventional medical video display systems that require practitioners to divert their attention away from the patient during procedures. There is a long-felt need for an AR eyewear system that enables continuous, real-time visualization of video feeds from diagnostic and surgical instruments without obstructing the practitioner's natural field of view. In addition, there is a long-felt need for a solution that reduces neck strain, eye fatigue, and ergonomic discomfort associated with traditional overhead or wall-mounted monitors. There is also a long-felt need for a display system that improves the ergonomics of performing medical procedures. Furthermore, there is a long-felt need for a wearable video display system that enhances procedural accuracy and safety by delivering anatomical and imaging data directly in the practitioner's field of view. Finally, there is a long-felt need in the art for a portable, wireless, and user-friendly AR system that supports a variety of medical imaging sources and is adaptable for both human and veterinary medicine.

The subject matter disclosed and claimed herein, in one embodiment, comprises a wireless augmented reality (AR) system that includes a pair of AR glasses configured with integrated heads-up displays (HUDs) embedded in semi-transparent optical lenses. Each HUD is driven by a high-resolution micro display embedded in the eyewear frame, capable of projecting real-time video content within the user's natural field of vision. The eyewear further includes an ambient light sensor for dynamic brightness adjustment, a nose bridge, and a pair of temples ergonomically designed for extended use in medical environments. A wired connection couples the AR glasses to a portable control unit which serves as an interface between the eyewear and a wireless video source. The control unit includes a microprocessor for decoding video data and a wireless receiver module for real-time streaming. The system is adapted to receive live video feeds from video-equipped medical instruments via a wireless HDMI transmitter.

In this manner, the wireless augmented reality system of the present invention provides a solution for the long-felt need for an ergonomic, immersive, and real-time visualization solution tailored for medical and veterinary professionals. The system enables continuous situational awareness by projecting imaging data directly into the user's line of sight, eliminating the need to look away from the patient or procedure. The AR system improves posture, reduces musculoskeletal strain, enhances procedural accuracy, and supports safer and more efficient medical interventions. The system provides medical practitioners with augmented reality glasses for use during surgical and interventional procedures, diagnostic tests, and more.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a wireless augmented reality eyewear system for use during a medical or veterinary procedure comprises an augmented reality (AR) eyewear and a control unit. The AR eyewear includes a pair of optical lenses, each lens incorporating a heads-up display (HUD) configured to present video data within a field of vision of a user. The AR eyewear further includes a pair of temples and a nose bridge that support the optical lenses. At least one micro display is embedded in the AR eyewear for projecting video content onto the HUD. The eyewear also comprises an ambient light sensor configured to automatically adjust the brightness of the HUD based on detected ambient lighting.

In one embodiment, the control unit comprises a wireless receiver module configured to receive a video signal from a wireless transmitter, a microprocessor configured to decode and format the video signal for display on the AR eyewear, a port (i.e., a USB-C port or a USB-micro B port) for supplying power and video data to the AR eyewear via a wired connection, and a rechargeable battery configured to power both the control unit and the AR eyewear. The system further includes a wireless transmitter configured to connect to a video source and wirelessly transmit the video signal to the control unit using a high-bandwidth wireless protocol.

In another aspect, a method of displaying medical video in an augmented reality system worn by a medical practitioner is disclosed and comprises the steps of powering on a portable control unit and connecting an AR eyewear device to the control unit via a connecting cable (i.e., USB-C cable). The method further includes wirelessly transmitting a video signal from a medical imaging device using a wireless transmitter and receiving the video signal at the control unit via a wireless receiver. The received video signal is then decoded and formatted using a microprocessor embedded in the control unit. The formatted video signal is projected onto a heads-up display (HUD) integrated into at least one lens of the AR eyewear. The method further comprises the step of adjusting the brightness of the HUD based on ambient light using an ambient light sensor integrated into the AR eyewear.

In another embodiment, an augmented reality eyewear device for medical visualization is disclosed and comprises a semi-transparent optical lens configured to transmit ambient light (i.e., at least 80 percent of ambient light). A micro display is embedded within the eyewear and is configured to project video content onto an inner surface of the optical lens. A heads-up display (HUD) is aligned with the wearer's natural field of view and is configured to superimpose anatomical data sourced from a medical imaging device. The eyewear further comprises a wired interface for receiving formatted video from a portable control unit, wherein the anatomical data includes representations of muscle tissue, nerve branches, tendons, or connective structures in real time.

In one embodiment, the system includes a gaze-based input mechanism configured to transmit a video feed or selection of a region of interest in the projected visual content based on a direction of the user's gaze.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of wireless augmented reality (AR) system of the present invention in accordance with the disclosed structure;

FIG. 2 illustrates a perspective view showing a medical professional wearing the AR eyewear and using the wireless augmented reality system of the present invention in accordance with the disclosed structure;

FIG. 3 illustrates a perspective view showing use of the eyewear being worn without compromising the wearer's point of view in accordance with one embodiment of the present invention;

FIG. 4 is a flow diagram illustrating a method of using a wireless augmented reality (AR) system during a medical procedure, in accordance with an embodiment of the present invention; and

FIG. 5 illustrates an enlarged view of AR glasses showing an AR video being projected in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a wireless augmented reality (AR) system that overcomes the limitations of conventional medical video display systems that require practitioners to divert their attention away from the patient during procedures. There is a long-felt need for an AR eyewear system that enables continuous, real-time visualization of video feeds from diagnostic and surgical instruments without obstructing the practitioner's natural field of view. In addition, there is a long-felt need for a solution that reduces neck strain, eye fatigue, and ergonomic discomfort associated with traditional overhead or wall-mounted monitors. There is also a long-felt need for a display system that improves the ergonomics of performing medical procedures. Furthermore, there is a long-felt need for a wearable video display system that enhances procedural accuracy and safety by delivering anatomical and imaging data directly in the practitioner's field of view. Finally, there is a long-felt need in the art for a portable, wireless, and user-friendly AR system that supports a variety of medical imaging sources and is adaptable for both human and veterinary medicine.

The present invention, in one exemplary embodiment, is a method of displaying a medical video in an augmented reality system worn by a medical practitioner and comprises the steps of powering on a portable control unit and connecting an AR eyewear device to the control unit via a connecting cable (i.e., a USB-C cable). The method further includes wirelessly transmitting a video signal from a medical imaging device using a wireless transmitter and receiving the video signal at the control unit via a wireless receiver. The received video signal is then decoded and formatted using a microprocessor embedded in the control unit. The formatted video signal is projected onto a heads-up display (HUD) integrated into at least one lens of the AR eyewear. The method further comprises the step of adjusting the brightness of the HUD based on ambient light using an ambient light sensor integrated into the AR eyewear.

Reference will now be made in detail to the present preferred embodiments of the invention, examples of which are illustrated in the accompanying drawings. Wherever possible, the same reference numerals are used in the drawings and the description to refer to the same or like parts.

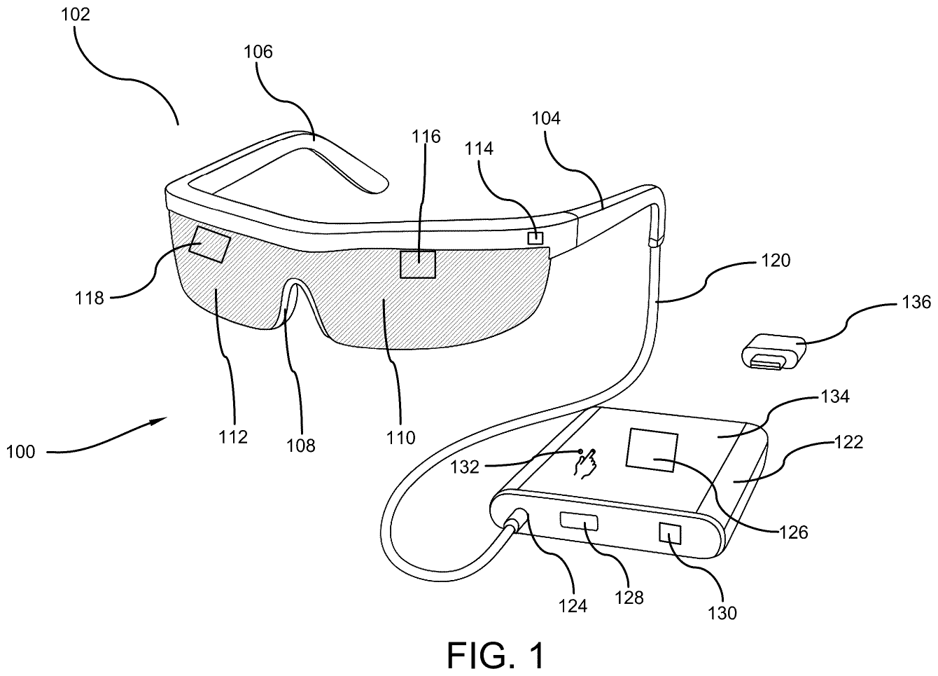

Referring initially to the drawings, FIG. 1 illustrates a perspective view of wireless augmented reality (AR) system of the present invention in accordance with the disclosed structure. The wireless augmented reality system 100 of the present invention is an innovative wireless AR device specifically designed for healthcare providers to improve ergonomics and efficiency during medical procedures. Specifically, the system 100 enables medical practitioners to view live video feeds from diagnostic and surgical instruments directly on an AR heads-up display (HUD) within their field of vision. The system 100 helps medical practitioners eliminate the need to look away from the patient or reposition themselves to view external monitors, thereby improving workflow, posture, and focus during procedures. The eyewear system 100 is particularly designed to be used during medical or veterinary procedures, including but not limited to colonoscopies, endoscopies, ultrasounds, arthroscopies, laparoscopies, and other minimally invasive diagnostics or surgeries.

The system 100 includes an AR eyewear 102 in the form of AR glasses that have integrated HUD. The eyewear 102 has a pair of temples 104,106 and a nose bridge 108 which together support a pair of optical lenses 110, 112, wherein each optical lens incorporates a HUD. The optical lenses 110, 112 are made of polycarbonate or optical-grade plastic and have a transparency of 80%-95% light transmission to preserve real-world vision. An ambient light sensor 114 is included for automatic brightness adjustment of the AR video based on ambient lighting.

Each lens includes a high-resolution micro display for providing visual output of medical video feed without occluding the surrounding real-world environment. As illustrated, the lenses 110, 112 have micro displays 116, 118 for efficient projection and alternatively, optical waveguide technology can also be used in the system 100. The eyewear 102 is adapted to project a 120-inch virtual screen in the wearer's line of sight.

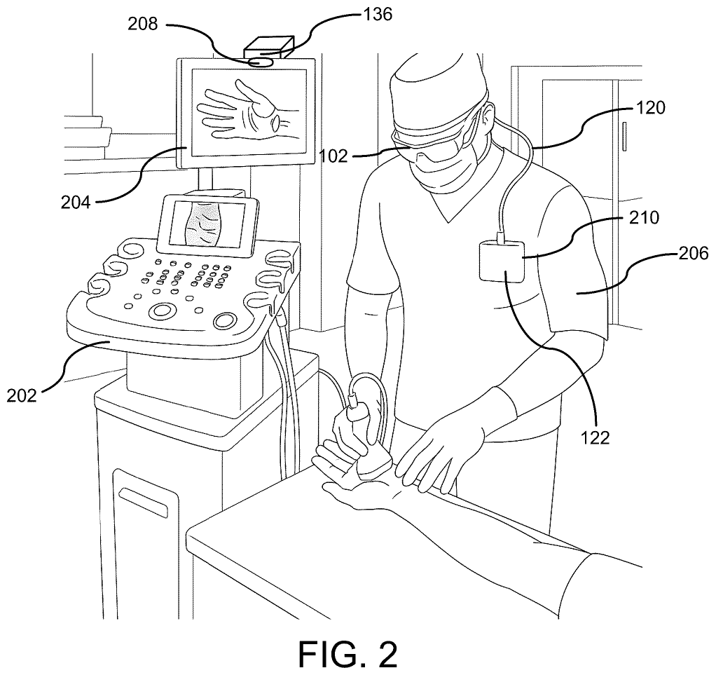

A wired connection 120, preferably a USB-C connection couples the eyewear 102 to a portable control unit 122. The portable control unit 122 functions as an interface hub between the AR glasses 102 and a video source such as a medical device. The portable control unit 122 includes a USB-C port 124 to connect to the eyewear 102 using the wired connection 120. The control unit 122 also includes an internal rechargeable battery 126 for providing electric power to the control unit 122 and the eyewear 102. The control unit 122 also includes a wireless receiver module 128 for wireless connectivity to the video source as described and illustrated in FIG. 2.

A microprocessor 130 embedded in the portable control unit 122 is adapted for real-time decoding of video signals received from the video source. Accordingly, the portable control unit 122 decodes and formats the video for display in the AR eyewear 102. A control interface 132 may be disposed on the outer surface 134 of the portable control unit 122 and can be in the form of physical buttons or touchpad controls for basic operation such as switching on and off of the control unit 122.

The system 100 also includes a wireless connector 136 such as an HDMI connector that is configured to be plugged into a video source's HDMI or any other similar port for wirelessly sending the live feed to the control unit 122 for decoding and formatting. The wireless connector 136 is configured to wirelessly transmit video data to the control unit 122 via a high-bandwidth protocol (i.e., Wi-Fi 5, 802.11ac) or proprietary low-latency RF.

FIG. 2 illustrates a perspective view showing a medical professional wearing the AR eyewear and using the wireless augmented reality system of the present invention in accordance with the disclosed structure. In the present embodiment, a medical device 202 having a video source 204 is used with the system 100 by a medical professional 206. The wireless connector 136 is preferably plugged into the HDMI port 208 of the video source 204 to send the live feed wirelessly to the control unit 122. The eyewear 102 is worn like a conventional eyewear by the medical professional 206 and the control unit 122 can be kept in a pocket 210, thereby eliminating need for physically holding the control unit 122 in a hand.

Upon receiving the live feed from the wireless connector 136, the control unit 122 decodes and formats the video for AR display and transmits the formatted video stream via wired connection 120 to the AR glasses/eyewear 102. It should be noted that the eyewear 102 projects slightly above the natural line of sight, so the medical professional 206 only needs to glance upward and not turn their head or look away. Resolution of the projection can be, for example, in the range of 1920×1080 pixels per eye.

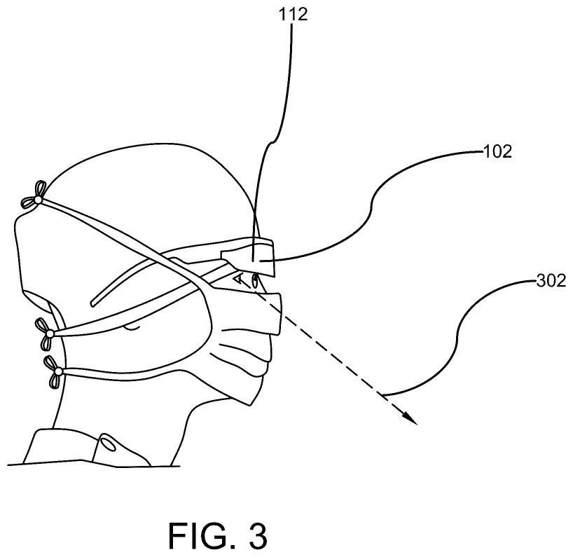

FIG. 3 illustrates a perspective view showing use of the eyewear being worn without compromising the wearer's point of view in accordance with one embodiment of the present invention. The eyewear 102 can be securely worn over a surgical cap and mask and is compatible with standard PPE (Personal Protective Equipment). The eyewear 102 does not obstruct the view of the medical practitioner 206 and the arrow 302 indicates that the wearer's natural line of sight is unobstructed and the AR data can be overlaid in the eyeglasses 110, 112.

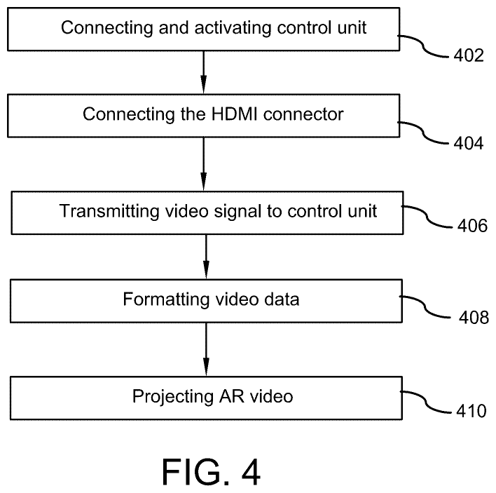

FIG. 4 is a flow diagram illustrating a method of using a wireless augmented reality (AR) system during a medical procedure, in accordance with an embodiment of the present invention. Initially, the portable control unit is powered, and a medical practitioner wears the AR eyewear 102 and connects the eyewear 102 to the control unit 122 via the wired connection 120 (Step 402). Then, the wireless HDMI transmitter 136 is connected to a video-equipped medical instrument, such as an ultrasound machine, colonoscope, or endoscope (Step 404). The wireless HDMI transmitter wirelessly transmits the video signal from the medical instrument to the control unit (Step 406) and the control unit 122 decodes and formats the received video data (Step 408). The formatted video signal is projected onto the heads-up display (HUD) embedded within the AR eyewear, presenting a virtual display within the user's natural field of view (Step 410). Using the system 100, the medical practitioner performs the intended medical procedure while continuously monitoring the live video feed through the AR display. The user can power off the devices and disconnects the system components on completion of a medical procedure.

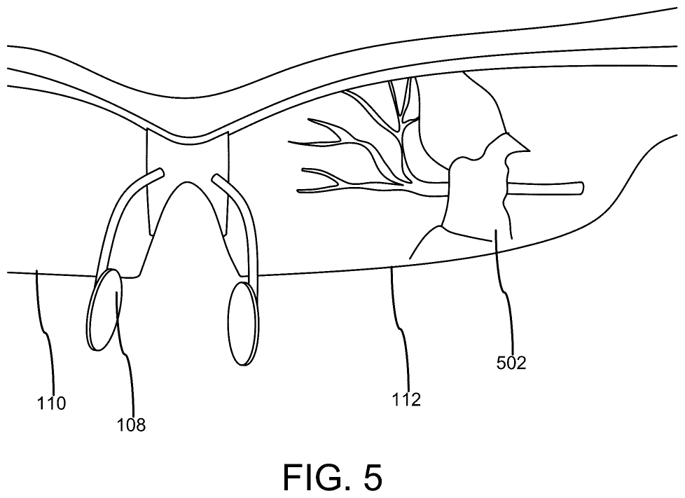

FIG. 5 illustrates an enlarged view of AR glasses showing an AR video being projected in accordance with the disclosed structure. As shown in FIG. 5, the system 100 includes a pair of transparent or semi-transparent optical lenses 110, 112 integrated into the AR eyewear 102. Each lens incorporates a heads-up display (HUD) that presents a detailed anatomical visualization 502 within the user's field of view. The HUD enables the practitioner to maintain focus on the patient while simultaneously receiving relevant visual data projected onto the lens. The HUD is implemented using micro displays (as shown in FIG. 1), wherein a high-resolution micro display embedded within the eyewear projects digital content onto the inner surface of the corresponding optical lens. The projection is aligned such that the virtual content appears superimposed over the real-world view, without obstructing the practitioner's vision.

The anatomical overlay 502, as illustrated, includes muscle tissue, nerve branches, tendons, and connective structures and may be sourced in real time from a connected diagnostic imaging device such as an ultrasound, MRI, or CT scanner via the HDMI connector.

In one embodiment of the present invention, the eyewear 102 may support voice commands and one or more voice commands can be used for capturing screenshots, adjusting brightness, and switching video feeds. Furthermore, the eyewear 102 may support gaze-based interaction for zooming into regions of interest for specific medical procedures.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “wireless augmented reality eyewear system”, “wireless augmented reality system”, “eyewear system”, and “system” are interchangeable and refer to the wireless augmented reality eyewear system 100 of the present invention.

Notwithstanding the forgoing, the wireless augmented reality eyewear system 100 of the present invention can be of any suitable configuration as is known in the art without affecting the overall concept of the invention, provided that it accomplishes the above stated objectives. One of ordinary skill in the art will appreciate that the wireless augmented reality eyewear system 100 as shown in the FIGS. are for illustrative purposes only, and that many other configurations of the wireless augmented reality eyewear system 100 are well within the scope of the present disclosure. Although the dimensions of the wireless augmented reality eyewear system 100 are important design parameters for user convenience, the wireless augmented reality eyewear system 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An augmented reality eyewear system for enhancing medical procedures comprising:
an eyewear system having an AR eyewear including AR glasses with integrated HUD, wherein said AR eyewear having a pair of temples and a nose bridge which together support a pair of optical lenses;
a wired connection; and
a portable control unit;
wherein each one of said pair of optical lenses incorporates a micro display in said HUD;
wherein said pair of optical lenses having a material selected from the group consisting of a polycarbonate and an optical-grade plastic;
wherein each said micro display providing a visual output of a medical video feed without occluding the surrounding real-world environment;
wherein said micro display embedded within said AR eyewear on an inner surface of each one of said pair of optical lenses;
wherein said wired connection couples said AR eyewear to said portable control unit; and
further wherein said portable control unit having a wireless interface between said AR eyewear and a video source of a medical device.

2. The augmented reality eyewear system of claim 1, wherein a wearer of said AR eyewear having an unobstructed point of view under said AR eyewear.

3. The augmented reality eyewear system of claim 2, wherein said unobstructed point of view is the wearer's natural line of sight.

4. The augmented reality eyewear system of claim 3 further comprising an ambient light sensor for automatic brightness adjustment of said micro display of said AR eyewear based on ambient lighting.

5. The augmented reality eyewear system of claim 3, wherein each said micro display projects a 120-inch virtual screen above the wearer's natural line of sight.

6. The augmented reality eyewear system of claim 3, wherein said pair of optical lenses having a transparency of 80%-95% light transmission.

7. The augmented reality eyewear system of claim 3, wherein said portable control unit having a connecting port to connect to said AR eyewear with said wired connection.

8. The augmented reality eyewear system of claim 3, wherein said portable control unit having an internal battery for providing electric power to said portable control unit and said AR eyewear.

9. The augmented reality eyewear system of claim 3, wherein said portable control unit having a wireless receiver module for said wireless interface to said video source.

10. The augmented reality eyewear system of claim 3, wherein said portable control unit having a microprocessor for real-time decoding of video signals received from said video source.

11. The augmented reality eyewear system of claim 3, wherein said portable control unit having a control interface for switching on and off of said portable control unit.

12. A method for enhancing medical procedures using augmented reality eyewear, the method comprising the steps of:
providing an eyewear system having an AR eyewear including AR glasses with integrated HUD, a wired connection, a portable control unit, wherein said AR eyewear having a pair of temples and a nose bridge together supporting a pair of optical lenses, wherein each one of said pair of optical lenses incorporates a micro display in said HUD, wherein said pair of optical lenses having a material selected from the group consisting of a polycarbonate and an optical-grade plastic;
providing a visual output of a medical video feed in each of said micro display without occluding the surrounding real-world environment;
coupling said AR eyewear to said portable control unit with said wired connection, wherein said portable control unit having a wireless interface between said AR eyewear and a video source of a medical device, further wherein said medical device selected from the group consisting of a colonoscope, an endoscope, an ultrasound, an arthroscope, a laparoscope, an MRI, and a CT scanner.

13. The method for enhancing medical procedures using augmented reality eyewear of claim 12, wherein a wearer of said AR eyewear having an unobstructed point of view under said AR eyewear.

14. The method for enhancing medical procedures using augmented reality eyewear of claim 13, wherein said unobstructed point of view is the wearer's natural line of sight.

15. The method for enhancing medical procedures using augmented reality eyewear of claim 14 further comprising an ambient light sensor for automatic brightness adjustment of said micro display of said AR eyewear based on ambient lighting.

16. The method for enhancing medical procedures using augmented reality eyewear of claim 15, wherein each said micro display projects a 120-inch virtual screen above the wearer's natural line of sight.

17. The method for enhancing medical procedures using augmented reality eyewear of claim 15, wherein said pair of optical lenses having a transparency of 80%-95% light transmission.

18. The method for enhancing medical procedures using augmented reality eyewear of claim 15, wherein said portable control unit having a wireless receiver module for said wireless interface to said video source.

19. The method for enhancing medical procedures using augmented reality eyewear of claim 15, wherein said portable control unit having a microprocessor for real-time decoding of video signals received from said video source.

20. An augmented reality eyewear system for enhancing medical procedures comprising:
an eyewear system having an AR eyewear including AR glasses with integrated HUD, wherein said AR eyewear having a pair of temples and a nose bridge which together support a pair of optical lenses;
a wired connection; and
a portable control unit;
wherein each one of said pair of optical lenses incorporates a micro display in said HUD;
wherein said pair of optical lenses having a material selected from the group consisting of a polycarbonate and an optical-grade plastic;
wherein each said micro display providing a visual output of a medical video feed without occluding the surrounding real-world environment;
wherein said wired connection couples said AR eyewear to said portable control unit;
wherein said portable control unit having a wireless interface between said AR eyewear and a video source of a medical device;
wherein a wearer of said AR eyewear having an unobstructed point of view under said AR eyewear;
wherein said unobstructed point of view is the wearer's natural line of sight; and
further wherein said medical device selected from the group consisting of a colonoscope, an endoscope, an ultrasound, an arthroscope, a laparoscope, an MRI, and a CT scanner.
